(19) United States
(12) Patent Application Publication
(10) Pub. No.: US 2008/0206347 A1
(43) Pub. Date: Aug. 28, 2008

(54) Pharmaceutical Formulations Comprising Active Pharmaceutical Principles Adsorbed on Titanium Dioxide Nanoparticles

(75) Inventors: Alberto Marra, Ariano Irpino (IT); Alessandro Dondoni, Ferrara (IT); Carlo Alberto Bignozzi, Ferrara (IT); Renato Canevotti, Concorezzo (IT); Negrisoli Gian Paolo, Bergamo (IT); Valeria Dissette, Adria (IT)

Correspondence Address: ROTHWELL, FIGG, ERNST & MANBECK, P.C., 1425 K STREET, N.W., SUITE 800, WASHINGTON, DC, 20005, US

(73) Assignee: FLAMMA S.P.A., CHIGNOLO D'LSOLA (IT)

(21) Appl. No.: 11/915,337

(22) PCT Filed: Apr. 12, 2006
(86) PCT No.: PCT/EP2006/003348

§ 371 Date: Feb. 20, 2008

Related U.S. Application Data
(60) Provisional application No. 60/684,532, filed on May 26, 2005.

(51) Int. Cl. A61K 9/16; A61K 31/40; A61K 31/675; A61K 31/66

(52) U.S. Cl. 424/498; 424/489; 514/423; 514/89; 514/103; 977/773

(57) ABSTRACT

The invention provides methods for the adsorption of an active pharmaceutical ingredient on titianium dioxide nanoparticles which can be orally ingested allowing the drug release into the intestine without causing side effects to the upper gastrointestinal region.

The present invention refers to pharmaceutical formulations comprising active pharmaceutical principles adsorbed on titanium dioxide nanoparticles.

The invention relates also to a process of adsorbing a drug on titanium dioxide nanoparticles through a direct interaction between the drug and titanium dioxide nanoparticles or via hydrophobic interactions assisted with fatty acids pre-adsorbed on the surface of titanium dioxide nanoparticles.

### BACKGROUND OF THE INVENTION

Nanoparticles have been studied extensively as particulate carriers in several pharmaceutical and medical fields (Sakuma S. et al., Adv Drug Del Rev 2001, 47: 21-37).

Titanium dioxide nanoparticles are known for instance from WO 01/42140, EP 1514845. In addition to the use as photocatalysts, carriers for catalysts and several other application in material science, titanium dioxide nanoparticles have been used as sunscreen formulations (US 2005249682) and for promoting plant growth (US 2005079977).

To the best of the Applicant's knowledge, the use of titanium dioxide nanoparticles as carrier for drugs in pharmaceutical formulations has never been reported.

### DESCRIPTION OF THE INVENTION

The invention relates to a process of adsorbing a drug on titanium dioxide nanoparticles through a direct interaction between the drug and the titanium dioxide nanoparticles or via a hydrophobic interaction with a fatty acid pre-adsorbed on the surface of titanium dioxide nanoparticles.

When adsorbed on titanium dioxide, the drug is no more, or only slightly, water soluble in the pH range 0-5 and can be desorbed from the titanium dioxide nanoparticles, becoming water soluble, in the pH range 6-14. This enables the preparation of new pharmaceutical forms which can be orally ingested allowing the drug release in the basic intestinal region with a minor release of the drug in the acidic gastrointestinal region, thus avoiding drug-induced gastro-esophageal injuries.

According to one embodiment of the invention, the drug is directly adsorbed on a titanium dioxide powder, where the dimension of the titanium dioxide nanoparticles have diameters in the 4-50 nm range (nm=nanometers).

According to an alternative embodiment of the invention, the drug is adsorbed on titanium dioxide nanoparticles precoated with fatty acids. In this case, the hydrophobic interaction between the long alkyl chains of the fatty acids, organized on the titanium dioxide nanoparticles, is weakened in the basic intestinal region.

Suitable fatty acids for coating the Titanium dioxide nanoparticles according to the invention include for instance C6-C30 linear or branched, optionally unsaturated, aliphatic or cycloaliphatic mono- or poly-carboxylic acids. Particularly preferred fatty acids are selected from Lauric, Myristic, Undecenoic, Pentadecanoic, Palmitic, Stearic, Arachidonic, Behenic and Lignoceric Acids.

The invention can be applied to a broad class of drugs containing carboxylic, phosphonic or boronic acid functions which can be directly adsorbed on the surface of TiO₂, as well as to drugs which can give an hydrophobic interaction with the long alkyl chains of fatty acids.

A particularly preferred class of drugs which may be advantageously used according to the invention is that of diphosphonic acids, including for instance alendronic, risedronic, pamidronic, chlodronic, neridronic, ibandronic, etidronic, mildronic, minodronic, zoledronic, cimadronic, tiludronic acids and salts thereof. This class of drugs, widely used in the treatment of osteoporosis and other bone diseases, is in fact known to have a poor gastric tolerability, as discussed for instance in WO 01/76577. This document discloses, as a possible solution to the above mentioned problem, the formulation of bisphosphonates with zwitterionic phospholipids.

Examples of hydrophobic drugs include HMG-CoA reductase inhibitors or statins (atorvastatin, pravastatin, lovastatin, simvastatin), steroids, angiotensin 2 antagonists, cyclosporine, tacrolimus, anti-fungal azoles such as fluconazole, itraconazole and the like, antivirals such as acyclovir, poorly soluble anti psychotic, anti epileptic, anti parkinsonian and other CNS drugs such as carbamazepine, fluoxetine, oxcarbazepine, Vitamins A, D and E or analogues such as isotretinoin, non-steroidal anti-inflammatory agents such as nimesulide, ibuprofen.

The TiO₂ nanoparticles in the common form of Anatase, optionally containing different amounts of Rutile, are particularly preferred, being possible to produce powders with nanoparticle dimensions in the 4-50 nm range.

The adsorption of the drug or of the fatty acid which will then interact with a selected compound can be carried out in a polar or a non polar solvent both protic or aprotic, independently from the protonation degree of the carboxylic, phosphonic or boronic functions. It is preferred that the drug contains such functional groups in their fully protonated form.

Suitable solvents include for instance alcohols, acetone, acetonitrile, water or mixtures thereof.

The adsorption of a water-soluble drug on the nanoparticles is carried out by mixing an aqueous solution of the drug with the nanoparticles and stirring the obtained suspension for a period of time from 6 to 72 hours, at temperatures ranging from 20 to 40° C.

The pre-coating of titanium dioxide nanoparticles with fatty acids is carried out by mixing a solution of a fatty acid in a solvent, for instance methanol or ethanol, with the nanoparticles and stirring the obtained suspension for a period of time from 6 to 72 hours, at temperatures ranging from 20 to 40° C.

From about 20 mg to about 100 mg of fatty acids may be accordingly adsorbed on 1.00 g of titanium dioxide nanoparticles. Desorption experiments at different pH showed no appreciable release of fatty acids from the nanocrystalline substrate. This fact allows to rule out that the adsorbed fatty acids may trap on the surface of TiO₂ the drug with its subsequent release after fatty acid desorption.

The adsorption of a water-insoluble or poorly water soluble drug on the nanoparticles is carried out by mixing a solution of the drug in an organic solvent with the nanoparticles pre-coated with fatty acids and stirring the obtained suspension for a period of time from 6 to 72 hours, at temperatures ranging from 20 to 40° C.

The weight ratio of titanium dioxide nanoparticles to a selected drug is not critical and may range within wide limits. Anyhow, it will generally be from 10 to 100 parts by weight of titanium dioxide nanoparticles per part of drug.

The titanium dioxide nanoparticles loaded with a selected drug may be formulated into suitable oral administration forms, optionally in admixture with usual excipients. Examples of suitable formulations include tablets, soft or hard gelatine capsules, granules, powders, pills.

The amount of the titanium dioxide particles will depend of course on the kind of adsorbed drug and will be easily determined by the skilled person according to the guidance reported in the following examples and on the basis of simple routine experiments.

The adsorbed drug is delivered to the intestinal region without appreciable release in the gastroesophageal region.

The invention is illustrated in more detail by the following examples.

### EXAMPLE 1

### Sodium Risedronate

Adsorption on TiO₂

The electronic absorption spectra of both protonated and deprotonated Sodium Risedronate are characterized by an intense band in the UV region that can be confidently assigned to π-π*transitions localized on the pyridine ring.

To a stirred solution of Sodium Risedronate (625 mg) in deionized water (100 ml) increasing amounts of nanocrystalline TiO₂ were added (ca. 20 nm nanoparticle size). The absorption spectra show that the amount of drug which can be adsorbed on the surface of the nanocrystalline substrate reaches the value of 31%. This limit probably corresponds to a total surface coverage of the nanoparticles.

190 mg of Sodium Risedronate were adsorbed at 25° C. on 3.00 g of TiO₂ nanoparticles having an average diameter of 20-25 nm.

Desorption from TiO₂

Desorption of Sodium Risedronate from TiO₂ at 25° C. was studied in aqueous solution at different pH values. The absorption spectra of the solutions at different pH obtained after a 2 h stirring show that the amount of Risedronate released to the solution increases with pH and that the amount of Risedronate released after 30 min at pH=8 is comparable to that released after 2 hours, i.e. 35% of the initial concentration on TiO₂.

Desorption of Sodium Risedronate from TiO₂ at pH 9, 36° C.

The desorption as function of time of Sodium Risedronate from TiO₂ was studied in aqueous solution at pH 9 and at 36° C.

Samples Preparation

To an aqueous solution containing 625 mg of Sodium Risedronate in 100 ml of deionized water 3.00 g of TiO₂ was added. The suspension was stirred for 72 h at room temperature and then centrifuged at 4000 rpm for 10 min. The solid was suspended twice in a pH 1 HCl solution, stirred for 20 min and centrifuged. The solid was finally dried at 40° C. under vacuum.

The spectrophotometric analysis showed that 190 mg of Sodium Risedronate were adsorbed by 3.00 g of TiO₂.

Nine separate 30 mg portions of TiO₂ adsorbed with Sodium Risedronate were suspended in 10 ml of pH 9 NaOH solutions and left stirring for different times in the range 1-120 min. During drug desorption the pH of the suspension was kept at the original value by small addition of NaOH solution. In all cases the concentration of desorbed drug was corrected for volume changes.

The results obtained indicate that, after 1 minute, 25% of the adsorbed Sodium Risedronate was immediately desorbed. The amount of drug released increased, almost linearly, up to a value of 36% in the next 60 min, followed by a constant release of 36% at longer times.

Based on desorption and kinetic studies it is possible to conclude that 250 mg of TiO₂ carrying 16 mg of Sodium Risedronate should allow to release in the intestine 6-9 mg of the drug with negligible release in the gastroesophageal region (the daily dose is 5-35 mg; the oral bioavailability is 0.63%).

### EXAMPLE 2

### Sodium Alendronate

A NMR-based method was developed since alendronate lacks the chromophoric group of risedronate. The NMR experiments were carried out in H₂O instead of D₂O (or other deuterated solvents) simply using the aqueous solutions obtained after removal of TiO₂ by centrifugation without concentration or further treatment. Only a small amount of C₆D₆ in a sealed capillary tube (reusable) inserted into the NMR tube was required to achieve the field lock. In the broad-band proton decoupled ³¹P-NMR spectrum of sodium alendronate in H₂O at 25° C. the signal corresponding to the two phosphonate groups appeared as a singlet at 19 ppm. To determine the concentration of sodium alendronate in aqueous solutions, a known amount of an internal standard was added before each NMR measurements, namely 1,3,5-triaza-7-phospha-adamantane, a water-soluble phosphine. Due to the different oxidation state of the phosphorous atoms in these two classes of compounds (R₃P vs. RP(O)(OH)₂), significant differences in the relaxation times and therefore in the corresponding signal integrals were found. However, upon careful adjustment of the NMR acquisition parameters, a very good agreement between the signal integrals and the actual molar ratio of known mixtures of sodium alendronate (two P atoms per molecule) and 1,3,5-triaza-7-phospha-adamantane (one P atom per molecule) in water was observed.

Adsorption on TiO₂

TiO₂ (25 nm, 1.00 g) was added to a solution of Sodium Alendronate trihydrated (207 mg, 0.64 mmol) in H₂O (50 ml) and the resulting suspension (pH 4.5) was vigorously stirred in the dark at 25° C. for 48 h, then centrifuged (4,000 rpm, 5 min). The cloudy supernatant was recovered, treated with solid NaCl (ca. 3 g), kept at room temperature for 2 h, and centrifuged (4,000 rpm, 5 min). To the resulting solution was added 1,3,5-triaza-7-phospha-adamantane and the concentration of residual sodium alendronate was estimated by ³¹P-NMR as described above.

The analysis showed that 71 mg (34% of the initial amount) of alendronate was adsorbed. We found that after 24 h stirring (instead of 48 h) at 25° C., 66 mg (32% of the initial amount) of alendronate was adsorbed. Upon repeated experiments (48 h, 25° C.) adsorption range was 26-34%.

Desorption from TiO₂

Sodium Alendronate trihydrated was adsorbed on TiO₂ as described above. After centrifugation, the recovered solid was dried at 40° C./0.1 mbar for 6 h. A suspension of this solid in 50 ml of aqueous HCl (pH 1.0) or NaOH (pH 9.0) was vigorously stirred at the stated temperature in a nitrogen atmosphere. The pH of the basic solution was constantly monitored and maintained at the initial level by addition of 0.05 M NaOH.

Desorption at pH 1.0, 2 h stirring at 25° C.: sodium alendronate was not detected by NMR analysis.

Desorption at pH 9.0, 30 min stirring at 25° C.: 8%

Desorption at pH 9.0, 2 h stirring at 25° C.: 12%

Desorption at pH 9.0, 6 h stirring at 37° C.: 22%

Upon repeated experiments (pH 9.0, 6 h, 37° C.) a 16-22% desorption range was found.

250 mg of TiO₂ carrying 16 mg of Alendronate should allow the release to the intestine of 4 mg of the drug.

### EXAMPLE 3

### Atorvastatin Calcium

Direct adsorption of Atorvastatin Calcium on titanium dioxide can be successfully obtained. Atorvastatin Calcium can also be adsorbed on titanium dioxide nanoparticles, pre-coated with long alkyl chains carboxylic acids which are commonly present in foods. Also with this second procedure the drug can be released in the basic intestinal region without appreciable release in the acidic gastrointestinal region.

Adsorption on TiO₂

The adsorption of Atorvastatin Calcium on Titanium Dioxide was performed by stirring, for 2 h, 200 mg of TiO₂ in 25 ml of a methanol solution containing dissolved 100 mg of the drug and evaporating the mixture to dryness at 40° C.

Desorption from TiO₂

In the desorption experiments, five 30 mg portions of TiO₂/Atorvastatin were stirred at 25° C., for 30 min, in 10 ml of aqueous solutions in the pH range 1-9, and filtered. The adsorption spectra of the filtered solution show that only 2% of Atorvastatin was released at pH 1, while 43% of the active principle was released at pH 9.

The experiment was then repeated at 36° C., keeping unchanged the other conditions. The analysis of absorption spectra of the filtered solutions showed no changes in the amount of Atorvastatin released at pH 1 (i.e. 2%) and 9 (i.e. 43%).

250 mg of TiO₂/Atorvastatin carrying 83 mg of Atorvastatin should allow the release of 36 mg of the drug in the intestinal region.

Adsorption of Atorvastatin Calcium on TiO₂ Precoated with 110-Undecenoic Acid

Coating of Titanium dioxide nanoparticles with the long chain carboxylic acid was performed by stirring in 50 ml of methanol 1.00 g of TiO₂ with 1 ml of pure 10-undecenoic acid for 24 h. The suspension was then filtered and the solid washed twice with 10 ml portions of methanol and then dried at 40° C. The Infrared spectrum of the solid product clearly shows that the long aliphatic chain carboxylic acid is adsorbed on TiO₂.

Relevant bands of the 10-undecenoic acid at ca 2924, 2853, 1709, 1530 and 1420 cm⁻¹ are also observed for the sample adsorbed on TiO₂.

Adsorption of Atorvastatin on TiO₂ nanoparticles pre-coated with 10-undecenoic acid (abbreviated as TiO₂/Und) was performed by stirring, for 1 h, a 500 mg amount of TiO₂/Und in 50 ml of a methanol solution containing dissolved 250 mg of Atorvastatin. The suspension was then evaporated under vacuum and the solid dried at 40° C. overnight.

Desorption of Atorvastatin from TiO₂/Und

Desorption of Atorvastatin from nanoparticles of TiO₂/Und/Atorvastatin is pH dependent.

The electronic absorption spectra, in the UV spectral region, of Atorvastatin in water at different pH values are characterized by an intense absorption band in the UV region with a maximum at 244 nm (the molar extinction coefficient in aqueous solution is 49500 μmol^{—1} cm⁻¹). No relevant changes of the spectral features are observed in the 1-9 pH range.

Desorption of Atorvastatin from solid samples of TiO₂/Und/Atorvastatin was performed as follows:

five 50 mg portions of TiO₂/Und with adsorbed Atorvastatin were stirred for 30 min at 25° C. in 25 ml of an aqueous solution at different pH, in the range 1-9, and filtered. The absorption spectra of the filtered solutions show a negligible desorption of the active principle in the pH range 1-3, while a maximum desorption of 79% is observed at pH 9.

Adsorption of Atorvastatin Calcium on TiO₂/StCOOH

Adsorption of Stearic acid (abbreviated as StCOOH) on Titanium Dioxide nanoparticles was performed by stirring overnight 500 mg of TiO₂ in 20 ml of an acetone solution containing 500 mg of StCOOH. The solid was washed with two 10 ml portions of acetone and dried at 40° C.

Analysis of the solid sample showed that 50 mg of StCOOH were adsorbed on 500 mg of TiO₂.

A 200 mg sample of TiO₂/StCOOH was suspended in 25 ml of a methanol solution containing 100 mg of Atorvastatin Calcium and stirred for 2 h. The mixture was rotary evaporated at 40° C. to dryness.

Desorption of Atorvastatin Calcium from TiO₂/StCOOH

Five 30 mg portions of TiO₂/StCOOH with adsorbed Atorvastatin were stirred for 30 min at 25° C. in 10 ml of an aqueous solution at different pH, in the range 1-9, and filtered. The absorption spectra of the filtered solutions show a negligible desorption of the active principle in the pH range 1-3, while a maximum desorption of 58% is observed at pH 9.

Experiments were also carried out at 36° C.:

Two portions of 30 mg of TiO₂/StCOOH/Atotvastatin were stirred for 30 min at 36° C. in 10 ml of aqueous solutions at pH 1 and pH 9, and filtered. The absorption spectra of the filtered solutions show a negligible desorption (2%) of the active principle at pH 1, while a maximum desorption of 64% is observed at pH 9.

Adsorption of Atorvastatin Calcium on TiO₂/ω³

Adsorption of Omega 3 fatty acids (abbreviated as ω³) on Titanium Dioxide nanoparticles was performed by stirring overnight 1.000 g of TiO₂ in 40 ml of an acetone solution containing 1.0 ml of ω³

The solid was washed with two 10 ml portions of acetone and dried at 40° C.

Analysis of the solid sample showed that a 19 mg amount of ω₃ was adsorbed on 1.000 g of TiO₂.

A 200 mg sample of TiO₂/ω³ was suspended in 25 ml of a methanol solution containing 100 mg of Atorvastatin Calcium and stirred for 2 h. The mixture was rotary evaporated at 40° C. to dryness.

Desorption of Atorvastatin Calcium from TiO₂/ω³

Two 30 mg portions of TiO₂/ω³/Atorvastatin were stirred at 25° C. for 30 min in 10 ml of aqueous solutions at pH 1 and 9 and filtered. The absorption spectra of the filtered solutions show a negligible desorption of the drug at pH 1, while a desorption of 36% is observed at pH 9.

The desorption of Atorvastatin from TiO₂/ω³ was repeated at 36° C. in the same conditions. The absorption spectra of the filtered solutions show a negligible desorption of the active principle at pH 1, while a desorption of 45% is observed at pH 9.

Adsorption of Atorvastatin Calcium on TiO₂/LrCOOH

Adsorption of Lauric acid (abbreviated as LrCOOH) on Titanium Dioxide nanoparticles, was performed by stirring overnight 1.000 g of TiO₂ in 20 ml of an acetone solution containing 1.00 g of LrCOOH. The solid was washed with two 10 ml portions of acetone and dried at 40° C.

Analysis of the solid sample showed that 140 mg of LrCOOH were adsorbed on 1.000 g of TiO₂.

A 200 mg sample of TiO₂/LrCOOH was suspended in 25 ml of an ethanol solution containing 100 mg of Atorvastatin Calcium and stirred for 2 h. The mixture was rotary evaporated at 40° C. to dryness.

Desorption of Atorvastatin Calcium from TiO₂/LrCOOH

Two 30 mg portions of TiO₂/LrCOOH with adsorbed Atorvastatin were stirred for 30 min at 36° C. in 10 ml of an aqueous solution at two different pH, 1 and 9, and filtered. The absorption spectra of the filtered solutions show a negligible desorption of the active principle at pH 1, while a maximum desorption of 80% is observed at pH 9.

Adsorption of Atorvastatin Calcium on TiO₂/MrCOOH

Adsorption of Myristic acid (abbreviated as MrCOOH) on Titanium Dioxide nanoparticles was performed by stirring overnight 1.000 g of TiO₂ in 20 ml of an acetone solution containing 1.00 g of MrCOOH. The solid was washed with two 10 ml portions of acetone and dried at 40° C.

Analysis of the solid sample showed that 110 mg of MrCOOH were adsorbed on 1.000 g of TiO₂.

A 200 mg sample of TiO₂/MrCOOH was suspended in 25 ml of an ethanol solution containing 100 mg of Atorvastatin Calcium and stirred for 2 h. The mixture was rotary evaporated at 40° C. to dryness.

Desorption of Atorvastatin Calcium from TiO₂/MrCOOH

Two 30 mg portions of TiO₂/MrCOOH with adsorbed Atorvastatin were stirred for 30 min at 36° C. in 10 ml of an aqueous solution at two different pH, 1 and 9, and filtered. The absorption spectra of the filtered solutions show a negligible desorption of the active principle at pH 1, while a maximum desorption of 65% is observed at pH 9.

Adsorption of Atorvastatin Calcium on TiO₂/PmCOOH

Adsorption of Palmitic acid (abbreviated as PmCOOH) on Titanium Dioxide nanoparticles, was performed by stirring overnight 1.000 g of TiO₂ in 20 ml of an acetone solution containing 1.00 g of PmCOOH. The solid was washed with two 10 ml portions of acetone and dried at 40° C.

Analysis of the solid sample showed that 90 mg of PmCOOH were adsorbed on 1.000 g of TiO₂.

A 200 mg sample of TiO₂/PmCOOH was suspended in 25 ml of a ethanol solution containing 100 mg of Atorvastatin Calcium and stirred for 2 h. The mixture was rotary evaporated at 40° C. to dryness.

Desorption of Atorvastatin Calcium from TiO₂/PmCOOH

Two 30 mg portions of TiO₂/PmCOOH with adsorbed Atorvastatin were stirred for 30 min at 36° C. in 10 ml of an aqueous solution at two different pH, 1 and 9, and filtered. The absorption spectra of the filtered solutions show a negligible desorption of the active principle at pH 1, while a maximum desorption of 72% is observed at pH 9.

Adsorption of Atorvastatin Calcium on TiO₂/BnCOOH

Adsorption of Behenic acid (abbreviated as BnCOOH) on Titanium Dioxide nanoparticles, was performed by stirring overnight 1.000 g of TiO₂ in 20 ml of an acetone solution containing 1.00 g of BnCOOH. The solid was washed with two 10 ml portions of acetone and dried at 40° C.

Analysis of the solid sample showed that a 100 mg amount of BnCOOH was adsorbed on 1.000 g of TiO₂.

A 200 mg sample of TiO₂/BnCOOH was suspended in 25 ml of an ethanol solution containing 100 mg of Atorvastatin Calcium and stirred for 2 h. The mixture was rotary evaporated at 40° C. to dryness.

Desorption of Atorvastatin Calcium from TiO₂/BnCOOH

Two 30 mg portions of TiO₂/MrCOOH with adsorbed Atorvastatin were stirred for 30 min at 36° C. in 10 ml of an aqueous solution at two different pH, 1 and 9, and filtered. The absorption spectra of the filtered solutions show a small desorption of the active principle at pH 1, while a maximum desorption of 56% is observed at pH 9.

TABLE

| | % desorption | % desorption |
|---|---|---|
| Fatty acid | pH 1 | pH 9 |
| 10-Undecenoic | 5% | 79% |
| acid | | |
| Lauric acid | 2% | 80% |
| Myristic acid | 1% | 65% |
| Omega 3 | 5% | 45% |
| Stearic acid | 1% | 64% |
| Palmitic acid | 5% | 72% |
| Behenic acid | 9% | 56% |

The results obtained by using TiO₂ nanoparticles pre-coated with 10-undecenoic acid show that Atorvastatin can be quantitatively trapped on the solid substrate without being appreciably released at pH values typical of the gastroesophageal region. Substantial release of the drug from the solid substrate occurs at higher pH, typical of the intestinal region, with a 67% drug release at pH 9. If the formulations with the different fatty acids are normalized to an amount of 250 mg, corresponding to 83 mg of Atorvastatin, the amount of drug which can be released in the intestinal region can be tuned as follows:

TiO₂/Und/Atorvastatin; 65 mg at 25° C.

TiO₂/StCOOH/Atorvastatin; 48 mg at 25° C. and 53 mg at 36° C.

TiO₂/ω³/Atorvastatin; 30 mg at 25° C. and 37 mg at 36° C.

The different amounts of Atorvastatin released at pH 9 are strongly dependent by the chemical nature of the fatty acid, indicating that interaction forces of different intensities between active principle and fatty acid are mutually exerted and compete with drug solvation and desorption from the functionalized nanomaterial.

1. Oral pharmaceutical formulations comprising as the active ingredient a drug adsorbed on titanium dioxide nanoparticles.

2. Formulations according to claim 1 wherein the drug contains carboxylic, phosphonic or boronic acid functions.

3. Formulations according to claim 2 wherein the drug is a bisphosphonate.

4. Formulations according to claim 3 wherein the bisphosphonate is alendronic, risedronic, pamidronic, chlodronic, neridronic, ibandronic, etidronic, mildronic, minodronic, zoledronic, cimadronic, tiludronic acids and salts thereof.

5. Formulations according to claim 1 wherein the drug is hydrophobic and the titanium dioxide nanoparticles are coated with fatty acids.

6. Formulations according to claim 5 wherein the hydrophobic drugs are selected from HMG-COA reductase, steroids, angiotensin 2 antagonists, cyclosporine, tacrolimus, anti-fungal azoles, antivirals such as acyclovir, poorly soluble anti-psychotic, anti-epileptic, anti-parkinsonian and CNS drugs, Vitamins A, D and E or analogues, non-steroidal anti-inflammatory agents.

7. Formulations according to claim 5 wherein the drug is atorvastatin.

8. Formulations according to claim 1 wherein the TiO2 nanoparticles consist of Anatase, optionally containing different amounts of Rutile, having nanoparticle dimensions from 4 to 50 nm.

9. Formulations according to claim 1 in form of tablets, capsules, granules, powders, pills.

10. Titanium dioxide nanoparticles as carriers for selective pH dependent delivery in the gastrointestinal tract of orally administered drugs.

11. A process for adsorbing a drug on titanium dioxide nanoparticles which comprises the mixing of a solution of the drug in water or organic solvent with the nanoparticles, either as such or pre-coated with fatty acids, and stirring the obtained suspension for a period of time from 6 to 72 hours, at temperatures ranging from 20 to 40° C.
